(19) United States
(12) Patent Application Publication
(10) Pub. No.: US 2007/0020245 A1
(43) Pub. Date: Jan. 25, 2007

(54) Composition for the treatment of arthrosis/arthritis, especially for treating joints

(76) Inventor: Katharina Trott, Hannover (DE)

Correspondence Address: SHLESINGER, ARKWRIGHT & GARVEY LLP, Suite 600, 1420 King Street, Alexandria, VA, 22314, US

(21) Appl. No.: 11/409,262

(22) Filed: Apr. 24, 2006

Related U.S. Application Data
(63) Continuation of application No. PCT/EP04/11903, filed on Oct. 21, 2004.

(30) Foreign Application Priority Data
Oct. 23, 2003 (DE) 103 49 722.6

(51) Int. Cl. A61K 35/32; A61K 9/14

(52) U.S. Cl. 424/93.7; 424/488

(57) ABSTRACT

The invention especially relates to a composition for the treatment of arthrosis/arthritis, especially for treating joints, said composition containing at least one carrier substance for receiving at least one anti-inflammatory agent and chondrogenetic cells, in addition to at least one anti-inflammatory agent, and chondrogenetic cells.

### CROSS-REFERENCE TO RELATED APPLICATIONS

This application is a continuation of application no. PCT/EP2004/011903, filed Oct. 21, 2004, which claims the priority of German application no. 103 49 722.6, filed 23 Oct. 2003, and each of which is incorporated herein by reference.

The invention relates to a composition for the treatment of arthrosis/arthritis, especially for treating joints, implantable articles for treating arthrosis/arthritis, especially for treating joints, and various uses, as well as a medication for treating arthrosis/arthritis and an arthrosis/arthritis treatment kit.

Injury to the cartilage, the knee or other joints frequently results from abnormally high mechanical stresses that deform the related tissue matrix. The loads applied on a joint can fracture the collagen network in the matrix and reduce the strength of the tissue.

Cartilage injuries are difficult to treat since particular cartilage has a limited capacity to regenerate from corresponding injuries. Type II collagen is the main structural protein of the extracellular matrix in articular cartilage. Type II collagen that is similar to other types of collagen consists of three collagen polypeptides that form a triple helix configuration. The polypeptides are entwined and have telopeptide regions at each end that connect the collagen peptides. Collagen matrices in their natural state have numerous cross-linked triple helices where the individual molecules have an approximate molecular weight of 300,000 Dalton. Type II collagen is almost exclusively found in animal cartilage, whereas other types of collagen are found in human skin, membranes and bone.

The excessive decomposition of type II collagen in the outer layers of cartilaginous joint surfaces is also caused by osteoarthrosis/arthritis. The collagen network is correspondingly weakened, fibrillation subsequently develops, and matrix substances such as proteoglycans are lost and may be completely displaced. Such fibrillation of weakened osteoarthritic cartilage can lead to the calcination of the cartilage in the subchondral bone (Kempson, G. E. et al., Biochim. Biophys. Acta 1976, 428, 741; Roth, V. and Mow, V. C., J. Bone Joint Surgery, 1980, 62 A, 1102; Woo, S. L.-Y. et al., in: Handbook of Bioengineering (R. Skalak and S. Shien Eds.), McGraw-Hill, New York, 1987, pp. 4.1-4.44).

A method for the regenerative treatments of cartilage would be useful for treating arthrosis/arthritis and could be performed in an early stage of joint damage which would reduce the number of patients who have to undergo involved treatments such as receiving an artificial joint. With such a treatment method, the number of patients with osteoarthrosis/osteoarthritis could be reduced.

Methods for growing and using chondrocytic cells have been described by Brittberg, M. et al. (New Engl. J. Med. 1994, 331, 889). Autologous transplants are also disclosed that grow cells using these methods. In addition, Kolletas et al. investigated the expression of cartilage-specific molecules such as collagens and proteoglycans under continued cell cultivation (J. Cell Science 1995, 108, 1991). They found that apart from morphological changes during cultivation in monolayer cultures (Aulthouse A. et al., In Vitro Cell Dev. Biol., 1989, 25, 659; Archer, C. et al., J. Cell Science 1990, 97, 361; Hanselmann, H. et al., J. Cell Science 1994, 107, 17; Bonaventure, J. et al., Exp. Cell Res. 1994, 212, 97), a comparison with suspension cultures tested by various scientists and grown with agarose spheres, alginate spheres or as spinner cultures reveals that such morphologies do not change the chondrocyte-expressed markers such as type II and IX collagens, and the large, aggregated proteoglycans, aggrecan, versican and compound proteins do not change (Kolletas, E. et al., J. Cell Science 1995, 108, 1991).

In addition, chondrocytic cells from donors were grown in vitro to form neocartilage which was implanted in animals (Adkisson et al., “A Novel Scaffold-Independent Neo-cartilage Graft for Articular Cartilage Repair,” ICRS 2nd Symposium of the International Cartilage Repair Society, Nov. 16-18, 1998). In addition, chondrocytic cells were seeded on the cartilage surface of osteochondral nuclei to achieve regenerate cartilage (Albrecht et al., “Circumferential Seeding of Chondrocytes: Towards Enhancement of Integrative Cartilage Repair,” ICRS 2nd Symposium of the International Cartilage Repair Society, Nov. 16-18, 1998). Surface-defects in knee joints have been treated with various cultivated chondrocytes (Stone et al., Operative Techniques in Orthopedics 7 (4), pp. 305-311, October 1997, and Minas et al., Operative Techniques in Orthopedics 7 (4), pp. 323-333, October 1997).

Membranes and specific properties have been described in the following references:

U.S. Pat. No. 5,837,278—Geistlich describes a collagen-containing membrane that is resorbable and used in guided tissue regeneration. The membrane has a fibrous surface that permits cell growth and a smooth, opposite surface that prevents cell adhesion. The membrane product is derived from a natural collagen membrane (from the skin or tendon of calves or piglets) and although treated, it is described as maintaining their natural structural features. The collagen is cleaned with alkaline agents to remove the fat from the collagen and to decompose substances. Then the cleaned collagen is acidified, washed, dried, defatted and optionally cross-linked. The fats are saponified. The membrane is described as containing approximately 95 weight percent native collagen.

PCT WO 96/25961—Geistlich et al. describes a matrix for reconstructing cartilage tissue consisting of type II collagen. When producing the matrix, cartilage is taken from an animal and frozen, comminuted, dehydrated, defatted, washed, and treated with alkaline materials. Non-collagen, alkaline-soluble proteins are denatured, destroyed, dissolved and eliminated. Dialysis and freeze-drying are suggested as possible treatment steps. The matrix material is pressed into a required shape and then sterilized.

U.S. Pat. No. 4,424,208—Wallace et al. describes an injectable collagen implant material that especially has linked atelopeptide collagen and reconstituted atelopeptide collagen fibers that are dispersed in a liquid carrier. The atelopeptide of collagen does not have native telopeptide cross-linking. In the method described in this patent, collagen from bovine and pig corium (subepithelial skin layer) is softened by being placed in a mild acid, depilated, comminuted by a physical process such as grinding, dissolved by being treated with an acid and a proteolytic enzyme, treated with an alkaline solution and freed from enzymes. The cross-linked gel form of collagen is formed by radiation-induced or chemically induced cross-linking such as by adding glutaraldehyde. During this process, the fibrous form of collagen is produced by neutralizing the solution with a buffer such as Na₂HPO₄. The amount of collagen in the injectable implant contains 5 to 30% fibrous collagen, and 70 to 98% of the cross-linked gel form of the collagen.

U.S. Pat. No. 4,488,911—Luck et al. describes the formation of collagen fibers that are free from the immunogenic telopeptide area of native collagen. The telopeptide region represents cross-linking points in the native collagen. The fibers that can be cross-linked are described as being useful for sponges, prostheses, films, membranes and suture material. In the described method, collagen obtained from tendons, skin and connective tissue from animals such as cows is disbursed in an acetic acid solution, passed through a meat grinder, treated with pepsin to split the telopeptides and dissolve the collagen, precipitated, dialyzed, cross-linked by the addition of formaldehyde, sterilized and lyophilized. In this method, the atelocollagen form of the collagen is obtained that is free from non-collagen proteins such as glycosaminoglycans and lipids. In addition, the collagen can be used as a gel to produce for example a membrane, a film or a sponge, whereby the degree of cross-linkage of the collagen is can be controlled to alter its structural properties.

U.S. Pat. No. 6,283,980—Vibe Hanson et al. describe a method for treating joint surface cartilage including the transplantation of chondrocytes in a suitable matrix to a surface to be treated , by means of forms of the surface to be treated, placing chondrocytes in a suitable matrix on the surface to be treated, and protecting the surface with a protective cap.

U.S. Pat. No. 6,379,367—Vibe Hanson et al. describes a cartilage repair structure that has a cell-free membrane with a porous and dense surface and a neighboring, porous chondrocyte surface of a membrane, as well as a method to manufacture a seeded low-density chondrocyte culture in vitro, including the seeding of chondrocyte cells in suspension material, in order to enable the cells to divide and differentiate.

PCT/1B00/01911—Muller et al. describes a resorbable, fleece-like implantable type II collagen-based matrix including reconstituted type II collagen that is formed from solubilized, articular tissue. In addition, an implantable article is described including a matrix and chondrocytes retained in the matrix, a method to produce the matrix, a method to treat cartilage defects by transplanting the implantable article onto the defect site, and a method to produce an implantable article.

The preparations known from the prior art up the present still do not have satisfactory properties for the treatment of osteoarthrosis/arthritis. The goal of this invention is therefore to present a composition that yields excellent results in the treatment of osteoarthrosis/arthritis. The object of the invention and the corresponding goal are solved by a composition according to claim 1, an implantable article according to claim 14, uses according to claims 17 to 20, an arthrosis/arthritis medication according to claim 21, and an arthrosis/arthritis treatment kit according to claim 22.

The composition according to the invention for treating arthrosis/arthritis, especially for treating joints, contains at least one carrier substance for receiving at least one anti-inflammatory agent and cartilage-building substances, at least one anti-inflammatory agent, and cartilage-building cells.

In the case of cartilage-building cells chondrocytes are especially suited, and the carrier substance may, in particular be collagen, the anti-inflammatory agent may. In particular, be hyaluronic acid, rhein, or especially diacetyl rhein.

In the composition according to the invention, the carrier substance contains at least one anti-inflammatory agent for fighting inflammation processes, as well as cartilage-building cells that counteract cartilage damage arising from arthrosis/arthritis by growing new cartilage cells. This ensures a lasting repair and also effectively suppresses the inflammation associated with arthrosis/arthritis that frequently almost totally prevents lasting regeneration.

It is advantageous when the carrier substance is resorbable in the body tissue and/or decomposable so that additional surgery is not necessary to remove the carrier substance after e.g. being implanted which makes it less invasive for the patient.

It is also advantageous when the carrier substance is designed as a mechanical support structure, especially like a fleece since such a structure has repeatedly proven itself in practice.

It is also advantageous when the carrier substance is based on collagen and/or polyglycols and/or polylactides since collagen in particular has excellent mechanical properties for the construction of a fleece-like support structure.

It has proven to be advantageous in practice when the collagen is essentially type I, III, I/III or II collagen. It is also advantageous when the collagen is a natural or reconstituted collagen.

Frequently, in practice, the collagen includes solubilized, articular (joint) cartilage tissue and/or solubilized tendon, peritoneum, pericardium, skin or mucous membranes since this is a relatively economical and proven method.

In addition, it is also advantageous when the anti-inflammatory agent is a substance from the group consisting of hyaluronic acid, sulfated hyaluronic acid, cyanidanol, rhein, especially diacetyl rhein, rhein derivatives that are formed by the acylation of rhein with ethyl acetic acid or propanoic acid, and ortho-(2,6-dichloranalino)-phenyl acetic acid, and (R,S)-2-(4-isobutylphenyl) propionic acid since these substances have proven to be highly effective in practice. Hyaluronic acid is advantageously obtained from rooster combs or streptomyces bacterial cultures that are proven in practice.

A highly elegant and hence advantageous embodiment includes a carrier substance which contains at least one cross-linking agent. The cross-linking agent is an inflammatory agent or the anti-inflammatory agent, especially when the cross-linking agent is a substance from the group consisting of cyanidanol, rhein, diacetyl rhein, and rhein derivatives that are formed by means of acylation of rhein with ethyl acetic acid or propionic acid, hyaluronic acid, sulfated hyaluronic acid, as well as glutaraldehyde and ethyl dimethylaminopropyl carbodiimide. The glutaraldehyde and the ethyl dimethylaminopropyl carbodiimide are compounds that function solely as cross-linking agents and have no anti-inflammatory effect. The ortho-(2,6-dichloranalino)-phenyl acetic acid and (R,S)-2-(4-isobutyl phenyl) propionic acid are compounds that only have an anti-inflammatory effect and do not function as cross-linking agents.

It has proven to be mechanically advantageous in practice to design the composition as a fleece for application to the joint.

The inventive advantageous properties according to the invention also apply to the implantable article according to the invention, especially in the form of a fleece for treating arthrosis/arthritis, especially of joints, with a composition according to the invention.

For the above-cited reasons, it is advantageous when the article is formed as a fleece that is, in particular, especially reversibly deformable to minimize any restriction of freedom of joint movement.

The composition (also termed “matrix” in the following, where the term “matrix” is frequently used for only the carrier substance) can be reconstituted collagen of type I, III, I/III and II, hyaluronic acid, or contain a mixture of reconstituted collagen and hyaluronic acid, or it can consist of them, or it can be a natural fleece such as animal skin, peritoneum, pericardium, or mucosa with absorbed hyaluronic acid. This natural and cross-linked matrix has a fleece-like consistency that, when loaded with cells, can be implanted in a site of the body for repairs (of damaged tissue, for example), and can be molded into the proper shape to be deposited at the corresponding site. In its uncross-linked form, the matrix has a liquid or gelatinous consistency. When it is in the form of a fleece, the matrix is reversibly deformable so that an implantable textile possessing the matrix can be manipulated to make implantation easier.

The reconstituted collagen matrix can for example be obtained by reconstituting previously solubilized animal tissue such as cartilage, tendon, peritoneum or mucosa of an animal such as a horse, pig, cow (or calf), goat, chicken, rabbit, mouse, rat or kangaroo.

The hyaluronic acid material can be obtained from various commercial sources (such as Hyalart®, Synvisc®, etc.). The molecular weight can vary within the entire range of obtainable hyaluronic acid.

The natural matrix is produced by purifying animal tissue such as peritoneum, tendon, pericardium, intestinal mucosa or skin according to methods in various publications (such as U.S. Pat. No. 5,837,278). In addition to the already published steps, the natural material is impregnated with hyaluronic acid of various molecular weights. In addition, the hyaluronic acid molecules are adhered to the natural membrane.

The utilized collagen material is solubilized by means of a physical and/or chemical treatment. The solubilization process includes treatment with various buffers to remove impurities and separate the solid and liquid phases. The physical treatment to separate solid and liquid phases can be centrifuging, and chemical treatment can be with a proteolytic enzyme that breaks the collagen linking.

Reconstitution means that the linkage between the variable regions along the collagen molecule is reestablished in uncross-linked forms of collagen. As a result, the collagen loses its liquid or gelatinous consistency and becomes solid with more structural integrity so that it can be used as a framework for the growth of the cells.

The solubilization removes dead cells, proteoglycans, glycosamine, glycans and other associated proteins and molecules from the collagen. The treatments effectively purify the collagen with a yield greater than 90%. The collagen can then be reconstituted to obtain sufficient structural stability for use as a framework by introducing cross linkage in the collagen, and it can then be lyophilized to form a fleece-like matrix on which the cells can grow and adhere.

Alternately, the matrix compositions can be formed from recombinant collagen. The essentially pure recombinant collagen is not cross-linked; however, it has telopeptide regions. It is soluble and can be formed into a fleece-like matrix.

The matrix can have two smooth surfaces, or one smooth and one rough surface. A smooth surface on the matrix hinders cell growth, whereas a rough surface promotes it.

The surface properties of the matrix can be changed with the slow addition of an alcohol, such as ethanol (in a 10-30% solution), to the lyophilization mixture. The formation of a smooth and rough surface can be substantially influenced by the lyophilization conditions.

Furthermore, the consistency of the matrix can vary from a liquid form or gelatinous form to a solid, fleece-like flexible form depending on its contact with a physiologically compatible thickener or a thickening or gelatinization agent, heat treatment, or a chemical reaction such as an enzymatic reaction (i.e. treatment with pepsin), or reaction with a cross-linking agent. The resulting matrix properties can be accordingly varied. The strength of the matrix (Fₘₐₓ in Newton) should be approximately 0.7 to approximately 1.3, and preferably approximately 1.0 Newton. In addition, the maximum elasticity (Fₘₐₓ) should be approximately 4.6% to approximately 5.6%, approximately 0.176 to approximately 0.184 N/mm², preferably approximately 5.1% and approximately 0.18 N/mm². These elasticity parameters reflect the strength of the utilized membrane.

The cross-linking agent can be an aldehyde-based biocompatible cross-linking agent, or a polyvalent aldehyde such as glutaraldehyde. Likewise, the cross-linking agent can be a bifunctional agent with two components that react with the support matrix. Examples of the components are aldehydes, ketones, acetals, half acetals and components that are useful for oxidative coupling such as phenolic groups, quinones, such as flavoids, carboxyl groups and activated carboxylic acids. Likewise, ethyl dimethylaminopropyl carbodiimide (EDC) can be used as the cross-linking agent.

Preferable cross-linking agents are chemical compounds that contain two reactive groups. The reactive groups accelerate the cross-linking by bridging amino acids such as lysine components on the telopeptide section. Particularly preferred cross-linking agents are cyanidanol, rhein and its derivatives such as especially diacetyl rhein that has an anti-inflammatory effect. Other preferred anti-inflammatory agents are NSAIDs, nonsteroidal anti-inflammatory drugs) such as diclofenac (ortho-(2,6-dichloranilino)-phenyl acetic acid) or ibuprofen ((R,S)-2-(4-isobutylphenyl) propionic acid). The type of cross-linking agent and its concentration are determined by evaluating the effect on the consistency and physical properties of the matrix and its physiological compatibility with the region in the body in which the matrix and the cells are to be implanted.

Cross-linking can be achieved by heating or irradiating the compound. In addition, heat or radiation can be used on the compound to increase the degree of cross-linking in a composition that is already chemically cross-linked, for example from the previous addition of an aldehyde-containing component. The heat-induced increase in cross-linkage can also cause the composition to be less gelatinous and more solid.

The matrix of the present invention is particularly useful e.g. for the human hip where conventional collagen membranes cannot be implanted. In the treatment of all solid joints, the matrix to be manipulated can be placed on the side of the joint and remain on the side. Likewise, the matrix can additionally hold types of cells such as osteocytes for treating bone damage together with a suitable growth stimulation enzyme.

A specific property of the matrix that makes it suitable for treating osteoarthrosis/arthritis is the amount of anti-inflammatory agent that it contains which is used as a cross-linking agent. These molecules are released into the joint after implanting the matrix while the matrix is being chemically decomposed, producing a lasting anti-inflammatory effect at the application site.

In regard to the incorporated medications, the matrix can be viewed as a new form of biochemically intelligent, slow-release system in which living (human) cells are incorporated in the matrix that help release the anti-inflammatory agent.

The gelatinous form of the matrix loaded with cells, such as chondrocyte cells can be used for incorporation, for example by injecting it into a solid joint or other areas where the insertion of a fleece-like matrix (in which the collagen molecules are already cross-linked) could be difficult or impossible.

Since the gelatinous matrix can be injected in a site of the body, the above-cited cross-linking agent can be simultaneously injected at the site to promote cross-linking in the collagen molecules, e.g. hyaluronic acid or e.g. collagen/hyaluronic acid molecules of the matrix. For this reason, the cross-linking reaction can occur in vivo after implanting the cell-loaded material. The cross-linking causes the matrix to become more rigid because of the increased structural integrity and stability at the site of implantation. Any possible biocompatible cross-linking agent can be used.

Chondrocytes that are autologous or homologous can be retained in the support matrix for the treatment of cartilage defects in the joint. Chondrocytes can be directly grown on the carrier matrix or be loaded in standard dishes and/or on the matrix before use (typically two or three days before use). The chondrocyte-loaded carrier matrix is introduced into the joint through an arthroscope or in minimally invasive or open surgery. Suitable allogeneic and xenogenic chondrocytic cells can be used to repair cartilage defects.

The cell-loaded matrix can be incorporated in various ways to trigger or stimulate a repair of a physical defect or injury using different devices for implanting. Some of these techniques and devices are found in U.S. patent application Ser. No. 09/373,952 submitted on Aug. 13, 1999, in U.S. provisional application No. 60/096,597 submitted on Aug. 14, 1998, and U.S. provisional application No. 60/146,683 submitted on Aug. 2, 1999. All of these publications are hereby incorporated herein by reference.

For this reason, this invention teaches conventional methods and systems for effectively repairing or treating defects of articular cartilage and bone, osteochondral defects, skin and wound defects, and defects of ligaments, menisci and vertebral disks. These methods and systems include the use of an implantable article that has a reconstituted cartilage-like matrix with cells such as chondrocytic cells. To this end, the carrier matrix of the implant is made from a material with sufficient physical integrity to achieve a stable shape over a certain period to enable cell growth before and after transplanting and to offer a system that is similar to the natural environment of the cells to optimize cell growth differentiation. Within two to three months, the matrix resorbable in a body is inspected to see if it has actually dissolved without leaving significant traces and if it has not induced toxic decomposition products. The term “resorbable” also includes processes that decompose the carrier matrix by natural biological processes and that eliminate the decomposed carrier matrix and associated decomposition products for example through the lymph vessels or blood cells. The resorption process releases the anti-inflammatory agents that are even in the matrix on or in a special embodiment (i.e., as a cross-linking agent) and thereby counteract the inflammatory process in the osteoarthritic joints. This is a very special embodiment of the composition according to the invention since the anti-inflammatory agent simultaneously serves as a cross-linking agent and is chemically solid and serves as an immanent component of the carrier substance; while the carrier substance is simultaneously decomposing, a correspondingly retarded release and action spectrum is enabled.

An essential feature of the present invention is the fact that in the treatment of arthrosis/arthritis, especially of joints, the composition according to the invention has a carrier substance possessing cartilage-building cells (especially chondrocytes) and at least one anti-inflammatory agent. The anti-inflammatory agent can be applied by purely physical, simple mechanical means which is the usual case for cartilage-building cells, as well as in the above-cited special, advantageous embodiment of constructing the carrier substance or mechanically reinforcing the carrier substance as a cross-linking agent and hence becoming a chemical part of the carrier substance so that, as mentioned, when the carrier substance is correspondingly decomposed, there is a particularly effective retarding release of the anti-inflammatory agent which is a particularly effective way to combat the inflammatory process occurring in arthrosis/arthritis in addition to the simultaneous build-up of the damaged cartilage by the cartilage building cells.

The following examples only serve to further explain the invention.

General example of producing a cross-linked matrix with type II collagen and hyaluronic acid (reference: PCT/I B00/01911)

In one embodiment, the method for producing the carrier matrix of the invention involves scraping off articular cartilage (joint cartilage) from the surface of the joint of a large mammal or another animal such as a horse, pig, cow, goat, chicken, or kangaroo. The scraped off cartilage is frozen and then ground, for example in a liquid nitrogen or liquid argon atmosphere. The cartilage is shock-frozen in such an atmosphere to obtain a powdered-like material. The typical weight of the ground cartilage is approximately 300 g wet weight.

The powder-like material is then defatted in one or more washings with a suitable solvent, such as an alcohol, ether, benzene, paratoluene, or other solvent with a high degree of solubility for fat molecules. For example, 300 mL ethanol (in a 70% solution) can be used. The defatting solution allows the fat molecules to be extracted from the cartilage. The resulting solution containing the fat molecules can then be dried, for example by evaporating it at room temperature, or drying it by heating it to a bit more than 50° C. until a solid is obtained.

The defatted cartilage solid is then dissolved in a suitable acidic buffer, such as 0.05 M sodium acetate buffer at a pH of 1.5. The obtained solution is then treated with a proteolytic enzyme such as aqueous pepsin (0.1 mg/mL), preferably at a low temperature, such as approximately +4° C. The enzyme treatment can be repeated up to 10 times to optimize the product yield; however, five repetitions were found to be sufficient in many cases.

The treated material is then centrifuged at a temperature between approximately 4° C. and 10° C., and at a rotation speed of approximately 10,000-20,000 rpm for approximately 30-60 minutes. This can be done several times to obtain a clean phase separation. Three such repetitions generally suffice.

If adequate phase separation is observed, the pellet is discarded, and the supernatant obtained in several vials from centrifuging is stored. Then the supernatant is precipitated using a suitable saline solution and a buffer with a neutral pH, such as potassium chloride (17.5 weight percent) in phosphate buffer (0.02 M KH₂PO₄, pH 7.4) that salts out the precipitate. The term “salts out” is meant as saturating a solution with a salt so that a solid precipitates from the salt solution. In a preferred embodiment, the supernatant is obtained from centrifuging, and the precipitate generated from the first centrifuging step is approximately 82 g, and the potassium chloride is prepared in an overall volume of 2 L of the phosphate buffer. Then the precipitate is centrifuged at a speed of approximately 30,000-100,000 rpm for approximately 30-60 minutes to further separate the phases and form a pellet. The obtained supernatant is discarded while the obtained pellet is resuspended in a suitable acidic buffer, such as 0.05% acetic acid (200-500 mL). The concentration of the cartilage material in the solution is generally more than 1 mg/mL.

An aliquot of hyaluronic acid is added. The molecular weight of the added material fluctuates between 15,000 and >6×10⁶ Dalton.

To prepare a matrix according to the invention as a physically integrated structure for subsequent handling and manipulation, cross-linking is required as illustrated in the above examples.

A biocompatible cross-linking agent is then added to the cartilage/collagen-containing solution. As discussed above, the specific cross-linking agent can be selected to obtain a certain consistency and physical properties for the matrix, i.e., the above-described strength and elasticity. The cross-linking agent is prepared in a suitable neutral buffer, e.g. 10-40 mL of 0.2 M NaCl/0.05 M tris-HCl, pH 7.4 at a concentration of 20-100 mg/mL.

The solution containing the cross-linked collagen/cartilage is then lyophilized to obtain a solid. The lyophilization can be repeated after rewashing with an aqueous solution such as 10-20 mL distilled water (depending on the size of the lyophilized collagen pellet) at a temperature between approximately 20° C. and a 60° C., preferably at approximately 25° C., and at a pressure of approximately 0.05 mbar. If the temperature rises within this range, the degree of cross-linkage and corresponding stability of the material increases. In contrast, the degree of cross-linkage and corresponding stability falls when the temperature drops within this range. A preferred embodiment of the invention contains several lyophilization steps and produces two smooth surfaces at the ends of the matrix. In addition to using a cross-linking agent and exposure to heat, radiation can be used to increase the cross-linkage of the collagen.

The lyophilization times vary depending on the volume of the solution to be lyophilized and the size of the obtained pellet. For example, 100 mL solution is lyophilized for approximately 36 hours to obtain a suitable, dry, pellet-shaped solid. The obtained solid is hygroscopic and does not contain more than approximately 20% water in its polymer structure. Depending on the color of the cross-linking agent, the color of the solid varies from bright white to yellow and reddish. The solid is a fleece-like material.

This fleece-like material can be mechanically pressed into sheets for use with cells as an implementation article. An example of a pressing machine suitable for such purposes is published in PCT/1B 00/01911. If the fleece-like material is pressed for approximately 24 hours between these surfaces, the fleece-like sheet becomes easier to handle and manipulate. In addition, the sheet is tear-resistant.

Alternatively, the pressing device can be any suitable device with a matte, smooth surface with sufficient weight to exert continuous pressure on the matrix material. The pressing device is preferably made of stainless steel, and other metals and materials can be used such as plastic, glass or ceramic with similar mechanical properties. In general, a weight of approximately 650-850 g, preferably approximately 735 g, is sufficient to press a fleece-like starting piece with a thickness of 5 to approximately 10 mm, and a surface area of approximately 19 mm. After pressing, the thickness of the fleece-like sheet is approximately 0.5-2.0 mm.

The matrix material is sterilized before it is used, for example with radiation (UV or gamma radiation), or with another sterilization method such as epoxide or ozone sterilization. Radiation sterilization is a preferred method to minimize the contact of the matrix material with external chemical quantities. This final process (radiation sterilization) additionally stabilizes the collagen carrier matrix material.

The carrier substance (matrix) allows the subsequent cultivation and growth of chondrocyte cells on the matrix when it is fully saturated with cell culture medium.

Chondrocytic cells should tightly adhere to the matrix or become integrated in it, and should retain their phenotype, or at least have a tendency to redifferentiate after transplantation. In addition, the obtained cell matrix biocomposite should be sufficiently mechanically stable for it to be handled in surgeries.

General Example of Manufacturing a Natural Matrix with Type I/III Collagen and Hyaluronic Acid (Reference: U.S. Pat. No. 6,379,367).

Chondrocytes are cultivated in minimal, essential culture media that contain HAM F12 and 15 mM Hepes buffer, and 5-7.5% autologous serum in a CO₂ incubator at 37° C. Other components of the culture medium can be used to cultivate the chondrocytes. These cells are trypsinated using trypsin EDTA for 5-10 minutes and counted using Trypan Blue viability stain in a Burker-Turk chamber. The cell count is adjusted to 7.5×10⁵ cells per mL.

A natural, commercially obtainable collagen I/III matrix from various manufacturers (such as MACI-Maix, Matricel, Chondro-gide and Bio-gide, Geistlich; SIS, DePuy; Colbar Membrane, Colbar) or other collagen matrices from companies such as Opocrin, Italy, Baxter, USA, and others is/are cut to a suitable size that fits the floor of the well in the NUNCLON™ cell culture plates. In this case, a circle with a diameter of approximately 4 cm (the size can differ however) is placed on the floor of the well under aseptic conditions.

The matrix is then impregnated with a 0.0001 mmol saturated solution of an anti-inflammatory agent such as diacetyl rhein, rhein, diclofenac, ibuprofen, and other suitable substances, and different solvents, preferably not limited to water, saline solution, DMF (dimethyl formamide), and DMSO (dimethyl sulfoxide). Other solvents can be used from the entire range of organic solvents including alcohols, ethers, esters, ketones and halogenated hydrocarbons. The impregnation time can vary from one second to several weeks.

After impregnation, the material is lyophilized. The aqueous solutions used for impregnation can also be directly removed, but the material will then probably have a lower concentration of the anti-inflammatory agent.

Approximately 5×10⁶ cells in 5 mL culture medium are directly added to the framework material and dispersed on the surface. The plate is incubated in a CO₂ incubator at 37° C. for three days. After this period, the chondrocytes have arranged themselves in clusters and have begun to grow on the carrier. They cannot be removed from the carrier by being rinsed with a medium or even by mechanically exerting a slight amount of pressure on the matrix.

Example 1 describes a method for preparing a carrier substance (matrix) (in this case, the composition corresponds to the carrier substance) in the composition according to the invention, and the growth, and retention of the chondrocytes on the matrix. Example 2 describes an additional method for producing the carrier substance in the composition according to the invention. Example 3 shows the growth and retention of chondrocytic cells on the matrix.

### EXAMPLE 1

For extraction of type II collagen, 300 g (wet weight) piglet cartilage is ground under liquid nitrogen into a powder and washed three times with 300 mL of a 70% ethanol solution to remove the fat from the collagen. The defatted collagen solution is then dried at room temperature. The obtained collagen solid is then dissolved in a 0.05 M potassium acetate solution (pH 1.5), and pepsin is added up to a final concentration of 0.1 mg/mL. The solution is then centrifuged at 4° C. for 16-20 hours at 20,000 rpm for 30 minutes. After a centrifuging cycle, the obtained supernatant (approximately 300 mL) is collected. The remaining pellet is resuspended in a 0.05 M sodium acetate solution, and treated with pepsin, stirred, and the centrifuging steps are repeated until most of the pellet has dissolved.

To precipitate a type II collagen-containing solid, the supernatants are collected (overall volume of approximately 500 mL), and solid KCl is added to obtain a final concentration of 17.5% KCl. Solid KH₂PO₄ is then added to obtain a final concentration of 0.02 M in an aqueous solution. The solution is then centrifuged for 30 minutes at 95,000 rpm, and the sediment (pellet) is redissolved in a 0.05% acetic acid solution and dialyzed twice for 16 hours against 5000 mL of a 0.05% acetic acid solution at 4° C.

An aldehyde-based biocompatible cross-linking agent is added to the dialyzed sample at a concentration of 20-100 mg/mL in 10-40 mL of a 0.2 M NaCl, 0.05 M Tris HCL solution at a pH of 7.4. The resulting solution is then lyophilized twice according to the above described general example to obtain a solid. The cross linkage is increased by UV radiation of the resultant solid for 12 hours. The irradiation increases the mechanical stability of the obtained type II collagen matrix that is a subsequently sterilized with x-rays (gamma radiation).

Chondrocytic cells are enzymatically freed from the knee joint cartilage of an adult sheep using collagenase and hyaluronidase (from Sigma, St. Louis, Mo.), and resuspended in culture medium (Ham's F12). The culture medium contains 12% fetal calf serum, 50 pL/mL penicillin/streptomycin, 50 pL/mL glutamine (all of the substances are obtainable from Biochrom KG, Berlin, Germany), 50 μL/mL unessential amino acids (Gibco BRL, Paisley, Scotland), and 2.3 mM MgCl₂. Cells are cultivated in an 80 cm² culture flask that is coated with 0.1% gelatin (Nalge Nunc, Rochester, New York), and incubated at 37° C. in a moist 5% CO₂ environment for 4-6 weeks. When these cells become confluent, they are trypsinated and seeded on the type II collagen matrix that was produced as described in the above example at a concentration of approximately 4×103 cells/mL.

Collagen membranes (matrices) are incubated for three days in the above-described medium in petri dishes. Unseeded membranes and chondrocyte cells that can grow on Thermanox plastic cover slips (Nunc, Rochester, N.Y.) are used as controls in the experiment.

The overall morphology of the dried type II collagen matrices has a fractured, paperlike appearance. The fleece does not shrink or dissolve during the culturing process. Unseeded type II collagen matrices viewed under a light microscope reveals a three-dimensional, porous architecture with various pore sizes. Cell detritus or lacunae can not be observed.

Chondrocytes grown on plastic surfaces reveal a dense monolayer of spinocellular, fibroblast-like cells with ovoid nuclei and a lack of intercellular contacts.

The chondrocytes seeded on type II collagen matrices form a membrane of multi-layer sheets of cells with various cells that are entangled with/encaptured by the connecting type II collagen fibers. The ultrastructure of these cells is more reminiscent of chondrocytes than of a fibroblast cell type. Most of the cells have irregular nuclei, a granular endoplasmic reticulum, and prominent golgi fields. Such cells have a large amount of glycogen and reveal narrow extruded vesicles in their surfaces.

A comparison of the above-described morphological differences between the cells grown on the plastic surfaces and those grown on the collagen type II matrix indicates that the chondrocytes that grew on the collagen type II matrices are redifferentiated.

The studies in this example show that in vitro production of autologous cartilage-like tissue can be established using type II collagen matrices in the composition according to the invention. The implantable articles according to the invention bear active chondrocyte cells with the potential of growing into cartilage and repairing cartilage defects. The mechanical stability of the article makes it easy to handle and quasi introduced into cartilage defects.

### EXAMPLE 2

300 g (wet weight) of articular cartilage (joint cartilage) scraped from calf joints is ground under liquid nitrogen to obtain a powder-like material. The fat is then removed from the powder by washing the powder three times with 300 mL 70% ethanol and drying it at room temperature. The solid is redissolved in 0.05 M sodium acetate buffer, pH =1.5, and repeatedly treated with pepsin (0.1 mg/mL) under cold conditions. The treatment is continued for 16-20 hours. After centrifuging three times at a temperature of 4° C.-10° C. at 10,000-20,000 rpm for approximately 30-60 minutes, the supernatant is collected and precipitated by adding potassium chloride (17.5%) in phosphate buffer. The precipitate is collected by centrifuging at 30,000-40,000 rpm for 45 minutes, and it is resuspended in 0.05% acetic acid at an approximate concentration >1 mg/mL.

The resultant solution is then dialyzed three times against five liters of diluted acetic acid (0.05%)(16 hours) to remove all excess salt. 10-40 mL of the cross-linking agent glutaraldehyde (prepared in 0.2 M NaCl/0.05 M Tris HCl, pH 7.4 at a concentration of 50 mg/mL) are added to the dialyzed sample. A homogenous solution is produced that is lyophilized, washed with distilled water, and then lyophilized once more.

The resultant fleece-like material is mechanically pressed into sheets using the above-described machine and sterilized with UV and x-ray (gamma) radiation. The sterilization process further stabilizes the collagen material.

### EXAMPLE 3

Chondrocytes are grown in a CO₂ incubator at 37° C. in a minimal essential culture medium that contains HAM's F12 and 15 mM Hepes buffer and 5-7.5% autologous serum. Other compositions of culture media such as calf serum can also be used for cultivating the chondrocytes. The cells are trypsinated using EDTA (trypsin: 2.5% solution without Ca²⁺and Mg²+ in phosphate-buffered saline solution (PBS), EDTA: 1% solution without Ca²+ and Mg²+ in PBS; the final solution contains 0.05% trypsin) (5 to 10 minutes), and is counted using Trypan Blue viability stain in a Burker-Turk chamber. The cell count is adjusted to 7.5×105 cells per mL.

A Bio-gide membrane (Geistlich, Switzerland) is impregnated with a 0.1 M solution of DAR (diacetyl rhein) in DMF (dimethyl formamide) or DMSO (dimethyl sulfoxide). The membrane is then washed five times with sterile PBS. The washing water is discarded. The membrane is then cut to a suitable size that fits the floor of the well in the NUNCLON™ cell culture plate. In this special case, a circle with a diameter of approximately 4 cm (other sizes are also possible) is placed on the floor of the well under aseptic conditions. Approximately 5×106 cells in 5 mL culture medium are directly added to the substrate and dispersed over the surface. The plate is incubated in a CO₂ incubator at 37° C. for three days.

At the end of the incubation period, the medium is decanted, and a cold, iced 2.5% glutaraldehyde solution is added as a fixative that contains 0.1 M sodium salt of dimethyl arsenic acid. The matrix is then stained with Safranin O for histological evaluation.

It can be observed that the chondrocytic cells that have arranged themselves in clusters start to grow on the carrier and cannot be removed from the carrier by being rinsed with a medium or even by mechanically exerting a slight amount of pressure on the matrix. These cells have hence apparently adhered to the matrix.

1-22. (canceled)

23. Composition for treating arthrosis/arthritis, comprising:
a) at least one carrier substance for receiving at least one anti-inflammatory agent and cartilage-building cells;
b) at least one anti-inflammatory agent; and
c) cartilage-building cells.

24. Composition according to claim 23, wherein:
a) the carrier substance can be at least one of resorbed and decomposed in body tissue.

25. Composition according to claim 23, wherein:
a) the carrier substance is configured as a mechanical support structure.

26. Composition according to claim 23, wherein:
a) the carrier substance includes collagen, hyaluronic acid, sulfated hyaluronic acid, and at least one polyglycols and polylactides.

27. Composition according to claim 26, wherein:
a) the collagen consists essentially of one of a typee I, III, I/III, and II collagen.

28. Composition according to claim 26, wherein:
a) the collagen is one of a natural and a constituted collagen.

29. Composition according to claim 26, wherein:
a) the collagen is formed of at least one of solubilized, articular cartilage tissue and solubilized tendon, peritoneum, pericardium, skin, and mucous membranes.

30. Composition according to claim 23, wherein:
a) the anti-inflammatory agent is a substance from the group of hyaluronic acid, sulfated hyaluronic acid, cyanidanol, rhein, and rhein derivatives that are formed by the acylation of rhein with ethyl acetic acid or propanoic acid, and ortho-(2,6-dichloranalino)-phenyl acetic acid, and (R,S)-2-(4-isobutyl phenyl) propionic acid.

31. Composition according to 30, wherein:
a) the hyaluronic acid is obtained from one of rooster combs and streptomyces bacterial cultures.

32. Composition according to claim 23, wherein:
a) the carrier substance includes at least one cross-linking agent.

33. Composition according to claim 32, wherein:
a) the cross-linking agent includes a further anti-ilammatory inflammatory agent.

34. Composition according to claim 32, wherein:
a) the cross-linking agent includes a substance from the group consisting of cyanidanol, rhein, diacetyl rhein, and rhein derivatives that are formed by acylation of rhein with ethyl acetic acid or propionic acid, hyaluronic acid, sulfated hyaluronic acid and glutaraldehyde and ethyl dimethylaminopropyl-carbodiimide.

35. Composition according to claim 23, wherein:
a) the composition is fleece-like.

36. Implantable article for treating arthrosis/arthritis having a composition according to claim 23.

37. Article according to claim 36, wherein:
a) the article is fleece-like.

38. Article according to claim 36, wherein:
a) the article is reversibly deformable.

39. Article according to claim 36, wherein:
a) the article is configured for use treating joints.

40. Use of a composition according to claim 23 for treating arthrosis/arthritis.

41. Use of a composition according to claim 40 for treating joints.

42. Use of an article according to claim 36 for treating joints.

43. Use of a composition according to claim 23 for producing a medication for treating arthrosis/arthritis.

44. Use of a composition according to claim 43 for producing a medication for treating joints.

45. Use of an article according to claim 36 to produce a medication for treating arthrosis/arthritis, especially of joints.

46. Arthrosis/arthritis medication, comprising:
a) a composition according to claim 23 and an article according to claim 36.

47. Arthrosis/arthritis treatment kit containing a composition according to claim 23 and an article according to claim 36.

48. Composition according to claim 32, wherein:
a) the cross-linking agent is the anti-inflammatory agent.

49. Composition according to claim 30, wherein:
a) the rhein includes diacetyl rhein.

50. Composition according to 26, wherein:
a) the hyaluronic acid is obtained from one of rooster combs and streptomyces bacterial cultures.

51. Composition according to claim 23, wherein:
a) the carrier substance consists of collagen, hyaluronic acid, sulfated hyaluronic acid, and at least one of polyglycols and polylactides.

52. Composition according to claim 23, wherein:
a) the carrier substance includes a mechanical support structure configured like a fleece.

53. Composition according to claim 23, wherein:
a) the composition is selected for treating at least one of arthrosis and arthritis of joints.
